(19) United States
(12) Patent Application Publication
(10) Pub. No.: US 2015/0031642 A1
(43) Pub. Date: Jan. 29, 2015

(54) COMBINATION TREATMENT OF ACUTE MYELOID LEUKEMIA AND MYELODYSPLASTIC SYNDROME I

(71) Applicants: Dorothea RUDOLPH, Vienna (AT); Tillmann TAUBE, Biberach an der Riss (DE)

(72) Inventors: Dorothea RUDOLPH, Vienna (AT); Tillmann TAUBE, Biberach an der Riss (DE)

(73) Assignee: BOEHRINGER INGELHEIM INTERNATIONAL GMBH, Ingelheim am Rhein (DE)

(21) Appl. No.: 14/340,016

(22) Filed: Jul. 24, 2014

Related U.S. Application Data
(60) Provisional application No. 61/858,802, filed on Jul. 26, 2013.

(51) Int. Cl. A61K 31/706; A61K 31/519

(52) U.S. Cl. CPC A61K 31/706; A61K 31/519
USPC 514/43

(57) ABSTRACT

The present invention relates to the use of Volasertib or a salt thereof or the hydrate thereof in combination with Decitabine or a salt thereof or the hydrate thereof for treating patients suffering from acute myeloid leukemia (AML) or myelodysplastic syndrome (MDS).

The present invention relates to the use of Volasertib or a pharmaceutically acceptable salt thereof or the hydrate thereof in combination with Decitabine or a pharmaceutically acceptable salt thereof or the hydrate thereof for treating patients suffering from acute myeloid leukemia (AML) and myelodysplastic syndrome (MDS).

### BACKGROUND OF THE INVENTION

Acute myeloid leukemia (AML), also known as acute myelogenous leukemia, is a cancer of the myeloid line of blood cells, characterized by the rapid growth of abnormal white blood cells that accumulate in the bone marrow and interfere with the production of normal blood cells. As an acute leukemia, AML progresses rapidly and is typically fatal within weeks or months if left untreated. AML is the most prevalent form of adult acute leukemia, particularly among older adults and is slightly more common in men than women. There is an estimated prevalence of 30,000 cases of AML in the US and 47,000 in the EU.

The incidence of AML increases with age with a median age at diagnosis of 67 years. The global incidence CAGR for AML out to 2013 is 1.4%. An aging population, along with an increased incidence of treatment-related AML in cancer survivors, currently accounting for 10-20% of all AML cases, is expected to drive the incidence of AML. In addition, there is some geographic variation in the incidence of AML. In adults, the highest rates are seen in North America, Europe, and Oceania, while adult AML is rarer in Asia and Latin America.

AML accounts for approximately 1.2% of all cancer deaths. The 5 year survival rates for AML are low, driven by therapy failure and patients relapsing. Among patients <65 the 5 year survival rate is 34.4%, among patients >65 it is only 5%.

The WHO classification of myeloid neoplasms and acute leukemia is the current standard for classification of AML and incorporates genetic abnormalities into diagnostic algorithms. This classification is done by examining the appearance of the malignant cells under light microscopy and by using cytogenetics and molecular genetics to characterize any underlying chromosomal abnormalities or genetic changes. The subtypes impact on prognoses, responses to therapy and treatment decisions.

Older patients with AML are biologically and clinically distinct from those who are younger. Although the prognosis of AML worsens with increasing age, older patients are generally considered as age 60 years or older. They are more likely to suffer early death and to exhibit therapeutic resistance. Increasing age is associated with factors predictive of early death, such as poor performance status or various comorbidities, and of treatment resistance (e.g. adverse cytogenetics, secondary AML or the MDR phenotype). A recent analysis suggested that while intensive chemotherapy can be delivered to older patients (≦70 years old) with AML, it may not be beneficial to most, and could be harmful to some. Indeed, this analysis showed that the prognosis of most patients (72%) aged 70 years or older with AML is poor with intensive chemotherapy, with an 8-week mortality of 36% and a median survival <6 months. Therefore, a substantial number of older AML patients are not considered for intensive treatment; available data indicate that about 70% of patients aged 65 to 74 and less than 30% of patients aged older than 74 receive intensive therapy at initial diagnosis of AML.

Based on current treatment guidelines decitabine is an established treatment option for older AML patients.

Myelodysplastic syndromes (MDS) are clonal hematopoietic stem-cell disorders characterized by ineffective hematopoiesis, peripheral-blood cytopenias, and increased tendency to progress to acute myeloid leukemia (AML). The age-adjusted incidence of MDS is 3.3 cases per 100,000 people, and this rate appears to be increasing. MDS is primarily a disease of older adults, the median age of patients with MDS is approximately 70 years. This patient population is frequently affected by other comorbid conditions, which often influences treatment decisions. Treatment of MDS is based on prognostic factors that predict survival and progression to AML. Currently, the treatment of patients with MDS is guided by the International Prognostic Scoring System (IPSS). This system stratifies patients into four groups: low, intermediate-1, intermediate-2, and high-risk, based on number of cytopenias, percentage of bone marrow blasts, and karyotype. Low risk and intermediate-1 risk are usually grouped together as lower-risk disease, whereas intermediate-2 risk and high risk are grouped together as higher-risk disease. The survival of patients with higher-risk MDS is significantly different than that of patients with lower-risk disease. Without intervention, median survival of higher-risk patients is close to 12 months. Survival of patients with lower-risk disease is more diverse and ranges from a few months (poor-prognosis, lower-risk disease) to more than a decade. Therefore, the objectives of therapy are different in lower-versus higher-risk disease. While in lower-risk MDS, the goal is to relieve symptoms, manage cytopenias, and minimize the need for transfusions [eg: erythropoiesis-stimulating agents (ESA) and growth factors (GF)], in higher-risk MDS, disease-modifying therapies directed to slowing progression to AML and improving survival are used. These disease modifying therapies include hypomethylating agents (HMA, as e.g. decitabine), intensive chemotherapy, and allogeneic stem cell transplantation (SCT), with SCT currently being the only known curative modality. Despite these treatment alternatives, the prognosis of patients with higher-risk MDS remains very poor owing to the disappointing activity of standard chemotherapy-based therapies, particularly those with therapy-related MDS, the eventual loss of response to HMA, and the restriction of allogeneic SCT to younger patients with an appropriate donor.

Treatment of higher-risk patients is dependent on whether they are considered to be candidates for intensive therapy (e.g., allogeneic SCT or intensive chemotherapy). Clinical features relevant for this determination include the patient's age, performance status, comorbidities, patient's preference and availability of suitable donor and caregiver. The access to allogeneic SCT is restricted to approximately 8% of patients with MDS, owing to advanced age, concomitant comorbidities and/or donor availability. For higher-risk patients who are not candidates for high-intensity therapy, the use of HMA is considered the standard of care.

The efficacy of chemotherapeutic agents can be improved by using combination therapies with other compounds and/or improving the dosage schedule. Even if the concept of combining several therapeutic agents or improved dosage schedules already has been suggested, there is still a need for new and efficient therapeutic concepts for the treatment of cancer diseases, which show advantages over standard therapies.

Volasertib is a highly potent and selective inhibitor of the serine-threonine Polo like kinase (Plk), a key regulator of cell-cycle progression. Volasertib is a second-generation dihydropteridinone derivative with distinct pharmacokinetic (PK) properties. The problem underlying this invention was to develop a combination treatment and improved dosage schedules for combination therapy of Volasertib and Decitabine in AML or MDS with maximal activity and limited toxicity.

Volasertib (I) is known as the compound N4-[trans-4-[4-(cyclopropylmethyl)-1-piperazinyl]cyclohexyl]-4-[[(7R)-7-ethyl-5,6,7,8-tetrahydro-5-methyl-8-(1-methylethyl)-6-oxo-2-pteridinyl]amino]-3-methoxy-benzamide,

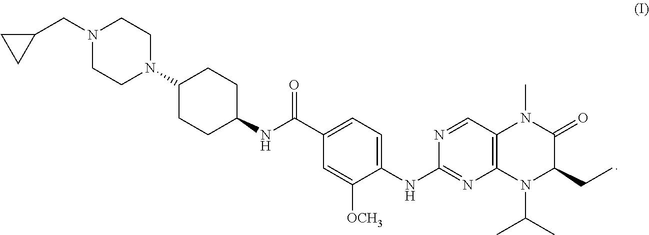

This compound is disclosed in WO 04/076454. Furthermore, trihydrochloride salt forms and hydrates thereof are known from WO 07/090844. They possess properties which make those forms especially suitable for pharmaceutical use. The above mentioned patent applications further disclose the use of this compound or its monoethanesulfonate salt for the preparation of pharmaceutical compositions intended especially for the treatment of diseases characterized by excessive or abnormal cell proliferation.

Document WO 2006/018182 discloses other combinations for the treatment of diseases involving cell proliferation.

Decitabine is a hypomethylating agent inhibiting DNA methyltransferases and known e.g. by the brand name Dacogen. Decitabine has been studied in the treatment of previously treated and untreated, young adult and older AML patients as well as treatment of patients with MDS including previously treated and untreated, de novo and secondary MDS, and intermediate-1, intermediate-2, and high-risk IPSS groups.

### SUMMARY OF THE INVENTION

In animal experiments it has been found that a cancer treatment with Volasertib and Decitabine comprise a synergistic efficacy profile (e.g. reduced tumor growth and beneficial side effect profile) compared to the mono therapy of both compounds.

Accordingly, a first object of the present invention refers to a pharmaceutical combination comprising Volasertib, optionally in the form of a pharmaceutically acceptable salt thereof or a hydrate thereof, and Decitabine, optionally in the form of a pharmaceutically acceptable salt thereof or a hydrate thereof, for simultaneous, separate or sequential use of the active ingredients.

Another object of the present invention relates to a kit comprising one pharmaceutical composition comprising Volasertib, optionally in the form of a pharmaceutically acceptable salt thereof or a hydrate thereof, and another pharmaceutical composition comprising Decitabine, optionally in the form of a pharmaceutically acceptable salt thereof or a hydrate thereof.

Another object of the present invention relates to a pharmaceutical kit, comprising a first compartment which comprises an effective amount of Volasertib and a second compartment which comprises Decitabine, optionally together with an instruction for administration of both active ingredients to a patient suffering from AML or MDS, wherein according to said instruction Volasertib (in one embodiment 250, 300, 350, 400, 450 or 500 mg, in another embodiment 300 or 350 mg) and Decitabine (in one embodiment 5 to 50 mg/m² BSA, in another embodiment 20 mg/m² BSA) is to be administered according to below mentioned dosage schedules.

Another object of the present invention relates to Volasertib, optionally in the form of a pharmaceutically acceptable salt thereof or a hydrate thereof, for use of treating AML or MDS, characterized in that Volasertib is administered in combination with Decitabine, optionally in the form of a pharmaceutically acceptable salt thereof or a hydrate thereof, wherein both active ingredients can be administered simultaneously, separately or sequentially.

Another object of the present invention relates to Decitabine, optionally in the form of a pharmaceutically acceptable salt thereof or a hydrate thereof, for use of treating AML or MDS, characterized in that Decitabine is administered in combination with Volasertib, optionally in the form of a pharmaceutically acceptable salt thereof or a hydrate thereof, wherein both active ingredients can be administered simultaneously, separately or sequentially.

Another object of the present invention relates to Volasertib, optionally in the form of a pharmaceutically acceptable salt thereof or a hydrate thereof, for use of treating AML or MDS characterized in that Volasertib is administered in combination with Decitabine, optionally in the form of a pharmaceutically acceptable salt thereof or a hydrate thereof, according to a dosage schedule (I) comprising or consisting of
a) administration of an effective amount of Volasertib or a pharmaceutically acceptable salt thereof or a hydrate thereof on minimally one day, preferably on two days, during a 4 week treatment cycle and
b) administration of an effective amount of Decitabine on at least one day of the said 4 week treatment cycle
   to a patient suffering from AML or MDS.

Another object of the present invention relates to Volasertib, optionally in the form of a pharmaceutically acceptable salt thereof or a hydrate thereof, for use of treating AML or MDS (dosage schedule (II)), characterized in that Volasertib is administered in combination with Decitabine, optionally in the form of a pharmaceutically acceptable salt thereof or a hydrate thereof, according to dosage schedule (I), wherein Volasertib or a pharmaceutically acceptable salt thereof or a hydrate thereof is administered on day 1 and on one of the days 7, 8, 9, 10, 11, 12, 13, 14, 15, 16, 17, 18, 19, 20 or 21 during a 4 week treatment cycle. Preferably, equal doses of Volasertib are administered on both days of administration.

Another object of the present invention relates to Volasertib, optionally in the form of a pharmaceutically acceptable salt thereof or a hydrate thereof, for use of treating AML or MDS (dosage schedule (III)), characterized in that Volasertib is administered in combination with Decitabine, optionally in the form of a pharmaceutically acceptable salt thereof or a hydrate thereof, according to one of the above dosage schedules (dosage schedule (I) or (II)) wherein in one embodiment 250 to 500 mg, in another embodiment 250, 300, 350, 400, 450 or 500 mg, yet in another embodiment 300 or 350 mg of Volasertib or a pharmaceutically acceptable salt thereof or a hydrate thereof are administered per day of administration.

Another object of the present invention relates to Volasertib, optionally in the form of a pharmaceutically acceptable salt thereof or a hydrate thereof, for use of treating AML or MDS (dosage schedule (IV)), characterized in that Volasertib is administered in combination with Decitabine, optionally in the form of a pharmaceutically acceptable salt thereof or a hydrate thereof, according to one of the above dosage schedules (dosage schedule (I), (II) or (III)) wherein Decitabine is administered on 5 days of the said 4 week treatment cycle, preferably from day 1 to 5.

Another object of the present invention relates to Volasertib, optionally in the form of a pharmaceutically acceptable salt thereof or a hydrate thereof, for use of treating AML or MDS (dosage schedule (V)), characterized in that Volasertib is administered in combination with Decitabine, optionally in the form of a pharmaceutically acceptable salt thereof or a hydrate thereof, according to one of the above dosage schedules (dosage schedule (I), (II) or (III)) wherein Decitabine is administered on 6 days of the said 4 week treatment cycle, preferably from day 1 to 6.

Another object of the present invention relates to Volasertib, optionally in the form of a pharmaceutically acceptable salt thereof or a hydrate thereof, for use of treating AML or MDS (dosage schedule (VI)), characterized in that Volasertib is administered in combination with Decitabine, optionally in the form of a pharmaceutically acceptable salt thereof or a hydrate thereof, according to one of the above dosage schedules (dosage schedule (I), (II) or (III)) wherein Decitabine is administered on 7 days of the said 4 week treatment cycle, preferably from day 1 to 7.

Another object of the present invention relates to Volasertib, optionally in the form of a pharmaceutically acceptable salt thereof or a hydrate thereof, for use of treating AML or MDS (dosage schedule (VII)), characterized in that Volasertib is administered in combination with Decitabine, optionally in the form of a pharmaceutically acceptable salt thereof or a hydrate thereof, according to one of the above dosage schedules (dosage schedule (I), (II) or (III)) wherein Decitabine is administered on 8 days of the said 4 week treatment cycle, preferably from day 1 to 8.

Another object of the present invention relates to Volasertib, optionally in the form of a pharmaceutically acceptable salt thereof or a hydrate thereof, for use of treating AML or MDS (dosage schedule (VIII)), characterized in that Volasertib is administered in combination with Decitabine, optionally in the form of a pharmaceutically acceptable salt thereof or a hydrate thereof, according to one of the above dosage schedules (dosage schedule (I), (II) or (III)) wherein Decitabine is administered on 9 days of the said 4 week treatment cycle, preferably from day 1 to 9.

Another object of the present invention relates to Volasertib, optionally in the form of a pharmaceutically acceptable salt thereof or a hydrate thereof, for use of treating AML or MDS (dosage schedule (IX)), characterized in that Volasertib is administered in combination with Decitabine, optionally in the form of a pharmaceutically acceptable salt thereof or a hydrate thereof, according to one of the above dosage schedules (dosage schedule (I), (II) or (III)) wherein Decitabine is administered on 10 days of the said 4 week treatment cycle, preferably from day 1 to 10.

Another object of the present invention relates to Volasertib, optionally in the form of a pharmaceutically acceptable salt thereof or a hydrate thereof, for use of treating AML or MDS (dosage schedule (X)), characterized in that Volasertib is administered in combination with Decitabine, optionally in the form of a pharmaceutically acceptable salt thereof or a hydrate thereof, according to one of the above dosage schedules (dosage schedule (I), (II), (Ill), (IV), (V), (VI), (VII), (VIII) or (IX)) wherein in one embodiment 5 to 50 mg/m² BSA, in another embodiment 20 mg/m² BSA of Decitabine are administered per day of administration.

Another object of the invention refers to a method of treating AML or MDS characterized in that Volasertib, optionally in the form of a pharmaceutically acceptable salt thereof or a hydrate thereof, and Decitabine, optionally in the form of a pharmaceutically acceptable salt thereof or a hydrate thereof, are administered according to one of the dosage schedules (I) to (X).

Another object of the invention refers to the use of Volasertib, optionally in the form of a pharmaceutically acceptable salt thereof or a hydrate thereof, for the manufacture of a medicament for treating AML or MDS in patients suffering from AML or MDS wherein the medicament is prepared for administration according to one of the dosage schedules (I) to (X).

Another object of the invention refers to the use of Decitabine, optionally in the form of a pharmaceutically acceptable salt thereof or a hydrate thereof, for the manufacture of a medicament for treating AML or MDS in patients suffering from AML or MDS wherein the medicament is prepared for administration according to one of the dosage schedules (I) to (X).

Another object of the invention is a pharmaceutical composition comprising an effective amount of Volasertib and an effective amount of Decitabine, optionally together with an instruction for administration of both active ingredients to a patient suffering from AML or MDS, wherein according to said instruction Volasertib is to be administered according to the above mentioned dosage schedules (I) to (X).

Another object of the present invention relates to Volasertib, optionally in the form of a pharmaceutically acceptable salt thereof or a hydrate thereof, for use of treating AML or MDS characterized in that Volasertib is administered in combination with Decitabine, optionally in the form of a pharmaceutically acceptable salt thereof or a hydrate thereof, according to a dosage schedule (XI) comprising or consisting of
a) administration of an effective amount of Volasertib or a pharmaceutically acceptable salt thereof or a hydrate thereof on minimally one day, preferably on two or three days, during a 6 week treatment cycle and
b) administration of an effective amount of Decitabine on at least one day of the said 6 week treatment cycle
   to a patient suffering from AML or MDS.

Another object of the present invention relates to Volasertib, optionally in the form of a pharmaceutically acceptable salt thereof or a hydrate thereof, for use of treating AML or MDS (dosage schedule (XII)), characterized in that Volasertib is administered in combination with Decitabine, optionally in the form of a pharmaceutically acceptable salt thereof or a hydrate thereof, according to dosage schedule (XI), wherein Volasertib or a pharmaceutically acceptable salt thereof or a hydrate thereof is administered on day 1 and on one of the days 5, 6, 7, 8, 9, 10, 11, 12, 13, 14, 15, 16, 17, 18, 19, 20, 21, 22, 23, 24, 25, 26, 27 or 28 during a 6 week treatment cycle. Preferably, equal doses of Volasertib are administered on both days of administration.

Another object of the present invention relates to Volasertib, optionally in the form of a pharmaceutically acceptable salt thereof or a hydrate thereof, for use of treating AML or MDS (dosage schedule (XIII)), characterized in that Volasertib is administered in combination with Decitabine, optionally in the form of a pharmaceutically acceptable salt thereof or a hydrate thereof, according to one of the above dosage schedules (dosage schedule (XI) or (XII)) wherein in one embodiment 200 to 500 mg, in another embodiment 200, 250, 300, 350, 400, 450 or 500 mg, yet in another embodiment 300 or 350 mg of Volasertib or a pharmaceutically acceptable salt thereof or a hydrate thereof are administered per day of administration.

Another object of the present invention relates to Volasertib, optionally in the form of a pharmaceutically acceptable salt thereof or a hydrate thereof, for use of treating AML or MDS (dosage schedule (XIV)), characterized in that Volasertib is administered in combination with Decitabine, optionally in the form of a pharmaceutically acceptable salt thereof or a hydrate thereof, according to one of the above dosage schedules (dosage schedule (XI), (XII) or (XIII)) wherein Decitabine is administered on 2 days of the said 6 week treatment cycle, preferably from day 1 to 2.

Another object of the present invention relates to Volasertib, optionally in the form of a pharmaceutically acceptable salt thereof or a hydrate thereof, for use of treating AML or MDS (dosage schedule (XV)), characterized in that Volasertib is administered in combination with Decitabine, optionally in the form of a pharmaceutically acceptable salt thereof or a hydrate thereof, according to one of the above dosage schedules (dosage schedule (XI), (XII) or (XIII)) wherein Decitabine is administered on 3 days of the said 6 week treatment cycle, preferably from day 1 to 3.

Another object of the present invention relates to Volasertib, optionally in the form of a pharmaceutically acceptable salt thereof or a hydrate thereof, for use of treating AML or MDS (dosage schedule (XVI)), characterized in that Volasertib is administered in combination with Decitabine, optionally in the form of a pharmaceutically acceptable salt thereof or a hydrate thereof, according to one of the above dosage schedules (dosage schedule (XI), (XII) or (XIII)) wherein Decitabine is administered on 4 days of the said 6 week treatment cycle, preferably from day 1 to 4.

Another object of the present invention relates to Volasertib, optionally in the form of a pharmaceutically acceptable salt thereof or a hydrate thereof, for use of treating AML or MDS (dosage schedule (XVII)), characterized in that Volasertib is administered in combination with Decitabine, optionally in the form of a pharmaceutically acceptable salt thereof or a hydrate thereof, according to one of the above dosage schedules (dosage schedule (XI), (XII) or (XIII)) wherein Decitabine is administered on 5 days of the said 6 week treatment cycle, preferably from day 1 to 5.

Another object of the present invention relates to Volasertib, optionally in the form of a pharmaceutically acceptable salt thereof or a hydrate thereof, for use of treating AML or MDS (dosage schedule (XVIII)), characterized in that Volasertib is administered in combination with Decitabine, optionally in the form of a pharmaceutically acceptable salt thereof or a hydrate thereof, according to one of the above dosage schedules (dosage schedule (XI), (XII) or (XIII)) wherein Decitabine is administered on 6 days of the said 6 week treatment cycle, preferably from day 1 to 6.

Another object of the present invention relates to Volasertib, optionally in the form of a pharmaceutically acceptable salt thereof or a hydrate thereof, for use of treating AML or MDS (dosage schedule (XIX)), characterized in that Volasertib is administered in combination with Decitabine, optionally in the form of a pharmaceutically acceptable salt thereof or a hydrate thereof, according to one of the above dosage schedules (dosage schedule (XI), (XII) or (XIII)) wherein Decitabine is administered on 7 days of the said 6 week treatment cycle, preferably from day 1 to 7.

Another object of the present invention relates to Volasertib, optionally in the form of a pharmaceutically acceptable salt thereof or a hydrate thereof, for use of treating AML or MDS (dosage schedule (XX)), characterized in that Volasertib is administered in combination with Decitabine, optionally in the form of a pharmaceutically acceptable salt thereof or a hydrate thereof, according to one of the above dosage schedules (dosage schedule (XI), (XII) or (XIII)) wherein Decitabine is administered on 8 days of the said 6 week treatment cycle, preferably from day 1 to 8.

Another object of the present invention relates to Volasertib, optionally in the form of a pharmaceutically acceptable salt thereof or a hydrate thereof, for use of treating AML or MDS (dosage schedule (XXI)), characterized in that Volasertib is administered in combination with Decitabine, optionally in the form of a pharmaceutically acceptable salt thereof or a hydrate thereof, according to one of the above dosage schedules (dosage schedule (XI), (XII) or (XIII)) wherein Decitabine is administered on 9 days of the said 6 week treatment cycle, preferably from day 1 to 9.

Another object of the present invention relates to Volasertib, optionally in the form of a pharmaceutically acceptable salt thereof or a hydrate thereof, for use of treating AML or MDS (dosage schedule (XXII)), characterized in that Volasertib is administered in combination with Decitabine, optionally in the form of a pharmaceutically acceptable salt thereof or a hydrate thereof, according to one of the above dosage schedules (dosage schedule (XI), (XII) or (XIII)) wherein Decitabine is administered on 10 days of the said 6 week treatment cycle, preferably from day 1 to 10.

Another object of the present invention relates to Volasertib, optionally in the form of a pharmaceutically acceptable salt thereof or a hydrate thereof, for use of treating AML or MDS (dosage schedule (XXIII)), characterized in that Volasertib is administered in combination with Decitabine, optionally in the form of a pharmaceutically acceptable salt thereof or a hydrate thereof, according to one of the above dosage schedules (dosage schedule (XI), (XII), (XIII), (XIV), (XV), (XVI), (XVII), (XVIII), (XIX), (XX), (XXI) or (XXII)) wherein in one embodiment 5 to 90 mg/m² BSA, in another embodiment 45 mg/m² BSA of Decitabine are administered per day of administration.

Another object of the invention refers to a method of treating AML or MDS characterized in that Volasertib, optionally in the form of a pharmaceutically acceptable salt thereof or a hydrate thereof, and Decitabine, optionally in the form of a pharmaceutically acceptable salt thereof or a hydrate thereof, are administered according to one of the dosage schedules (XI) to (XXIII).

Another object of the invention refers to the use of Volasertib, optionally in the form of a pharmaceutically acceptable salt thereof or a hydrate thereof, for the manufacture of a medicament for treating AML or MDS in patients suffering from AML or MDS wherein the medicament is prepared for administration according to one of the dosage schedules (XI) to (XXIII).

Another object of the invention refers to the use of Decitabine, optionally in the form of a pharmaceutically acceptable salt thereof or a hydrate thereof, for the manufacture of a medicament for treating AML or MDS in patients suffering from AML or MDS wherein the medicament is prepared for administration according to one of the dosage schedules (XI) to (XXIII).

Another object of the invention is a pharmaceutical composition comprising an effective amount of Volasertib and an effective amount of Decitabine, optionally together with an instruction for administration of both active ingredients to a patient suffering from AML or MDS, wherein according to said instruction Volasertib is to be administered according to the above mentioned dosage schedules (XI) to (XXIII).

The above described pharmaceutical combinations, pharmaceutical compositions, pharmaceutical kits, dosage schedules and other embodiments can be applied to patients of all ages, preferably to patients being older than 60 years, more preferably older than 65 years.

### BRIEF DESCRIPTION OF THE FIGURES

FIG. 1 shows the tumor growth kinetics in a nude mouse xenograft model derived from human AML cell line MV4;11. Tumor-bearing mice were treated for 3 weeks either with vehicle or with 10 mg/kg Volasertib (BI 6727) once a week i.v., 1.25 mg/kg decitabine twice a week i.p. or a combination of Volasertib and decitabine. Median tumor volumes are plotted over time. Day 1 was the first day, day 21 the last day of the experiment. Efficacy results from this xenograft models are considered valid for AML as well as MDS.

FIG. 2 shows the change of body weight over time in a nude mouse xenograft model derived from human AML cell line MV4;11. Tumor-bearing mice were treated for 3 weeks either with vehicle or with 10 mg/kg Volasertib once a week i.v., 1.25 mg/kg decitabine twice a week i.p. or a combination of Volasertib and decitabine. Median changes in body weight compared to day 1 are plotted over time. Day 1 was the first day, day 21 the last day of the experiment.

FIG. 3 shows the tumor growth kinetics in a nude mouse xenograft model derived from human AML cell line MV4;11. Tumor-bearing mice were treated for 3 weeks either with vehicle or with 10 mg/kg Volasertib once a week i.v., 2.5 mg/kg decitabine twice a week i.p. or a combination of Volasertib and decitabine. Median tumor volumes are plotted over time. Day 1 was the first day, day 21 the last day of the experiment. Efficacy results from this xenograft models are considered valid for AML as well as MDS.

FIG. 4 shows the change of body weight over time in a nude mouse xenograft model derived from human AML cell line MV4;11. Tumor-bearing mice were treated for 3 weeks either with vehicle or with 10 mg/kg Volasertib once a week i.v., 2.5 mg/kg decitabine twice a week i.p. or a combination of Volasertib and decitabine. Median changes in body weight compared to day 1 are plotted over time. Day 1 was the first day, day 21 the last day of the experiment.

FIG. 5 shows the tumor growth kinetics in a nude mouse xenograft model derived from human AML cell line MV4;11. Tumor-bearing mice were treated for 3 weeks either with vehicle or with 20 mg/kg Volasertib once a week i.v., 1.25 mg/kg decitabine twice a week i.p. or a combination of Volasertib and decitabine. Median tumor volumes are plotted over time. Day 1 was the first day, day 21 the last day of the experiment. Efficacy results from this xenograft models are considered valid for AML as well as MDS.

FIG. 6 shows the change of body weight over time in a nude mouse xenograft model derived from human AML cell line MV4;11. Tumor-bearing mice were treated for 3 weeks either with vehicle or with 20 mg/kg Volasertib once a week i.v., 1.25 mg/kg decitabine twice a week i.p. or a combination of Volasertib and decitabine. Median changes in body weight compared to day 1 are plotted over time. Day 1 was the first day, day 21 the last day of the experiment.

### DETAILED DESCRIPTION OF THE INVENTION

In case Volasertib is administered on minimally two days during a 4 week treatment cycle, then Volasertib is administered on two non-consecutive days during a 4 week treatment cycle.

The administration of an effective amount of Decitabine on at least one day of the said 4 week treatment cycle means that during the 4 week treatment cycle in which Volasertib is adminidstered minimally one time, also Decitabine is administered on at least one day.

The administration of Volasertib on day 1 and 15 during a 4 week treatment cycle means that one dosage of Volasertib or a pharmaceutically acceptable salt or a hydrate thereof is administered on day one and the second dosage is administered on day 15 to the patient suffering from AML or MDS in the four week treatment cycle.

The administration of Decitabine from days 1 to 5, days 1 to 6, days 1 to 7, days 1 to 8, days 1 to 9 or from days 1 to 10, respectively, during a 4 week treatment cycle means that a daily dosage of Decitabine or a pharmaceutically acceptable salt thereof is administered to the patient suffering from AML or MDS beginning on day one and ending with the last dosage on day 5, on day 6, on day 7, on day 8, on day 9 or on day 10, respectively, in the four week treatment cycle.

Accordingly, a complete four week treatment cycle according to one of the above mentioned dosage schedules may comprise the following administrations:
Day 1: one dosage of Volasertib (e.g. 300 or 350 mg) and one dosage of Decitabine (e.g. 20 mg/m² BSA);
Day 2 to day 5 (including): one dosage of Decitabine (e.g. 20 mg/m² BSA) per day;
Day 6 to day 14 (including): no administration of Volasertib and Decitabine;
Day 15: one dosage of Volasertib (e.g. 300 or 350 mg);
Day 16 to day 28 (including): no administration of Volasertib and Decitabine.

In case of the 6 week treatment cycle above mentioned explanations can be applied accordingly.

The treatment cycles can be repeated as long as patients are eligible for repeated cycles, i.e. until progression of disease and as long as neither patient nor investigator requests treatment discontinuation.

The instruction for coadministration may be in any form suitable for pharmaceuticals, e.g. in form of a leaflet added to the dosage form within secondary packaging or an imprint on the primary or secondary packaging.

Dosages/Volasertib:

For intraveneous treatment Volasertib may be administered to the human patient in a daily dose of 250 to 500 mg/application during the 4 week treatment cycle, in another embodiment 250, 300, 350, 400, 450 or 500 mg/application, yet in another embodiment 300 or 350 mg/application. For instance, Volasertib can be administered as a slow intravenous infusion over several hours, e.g. over about 1, 2, 4, 6, 10, 12 or 24 hours, preferably about 1 or 2 hours.

For intraveneous treatment Volasertib may be administered to the human patient in a daily dose of 200 to 500 mg/application during the 6 week treatment cycle, in another embodiment 200, 250, 300, 350, 400, 450 or 500 mg/application, yet in another embodiment 300 or 350 mg/application. For instance, Volasertib can be administered as a slow intravenous infusion over several hours, e.g. over about 1, 2, 4, 6, 10, 12 or 24 hours, preferably about 1 or 2 hours.

Dosages/Decitabine:

Decitabine may be administered in a total daily dose of 5 to 50 mg/m² BSA during the 4 week treatment cycle, e.g. in a total daily dose of 5, 10, 15, 20, 25, 30, 35, 40, 45 or 50 mg/m² BSA one or two times daily. The total daily dose may also be divided into two or three subdoses to be taken within one day. Preferably, the daily dose is administered in a single dose of 20 mg/m² BSA.

Decitabine may be administered in a total daily dose of 5 to 90 mg/m² BSA during the 6 week treatment cycle, e.g. in a total daily dose of 5, 10, 15, 20, 25, 30, 35, 40, 45, 50, 55, 60, 65, 70, 75, 80, 85 or 90 mg/m² BSA. The total daily dose may also be divided into two or three subdoses to be taken within one day. Preferably, the daily dose of 45 mg/m² BSA is administered in three doses of 15 mg/m² BSA (administration every 8 hours).

However, it may optionally be necessary to deviate from the dosage amounts specified for Volasertib and Decitabine, depending on the body weight or method of administration, the individual response to the medication, the nature of the formulation used and the time or interval over which it is administered. Thus, in some cases, it may be sufficient to use less than the minimum quantity specified above, while in other cases the upper limit specified will have to be exceeded. When large amounts are administered it may be advisable to spread them over the day in a number of single doses.

Dosage Forms and Formulation Aspects

Regarding any aspects of the invention for Volasertib pharmaceutically acceptable salts or hydrates thereof may be used, preferably trihydrochloride salt forms and hydrates thereof as disclosed in WO 07/090844. Dosages or amounts of the active ingredient provided in the context of this invention refer in any case to the free base equivalent, that is Volasertib in the free base form.

The term “therapeutically effective amount” shall mean that amount of a drug or pharmaceutical agent that will elicit the biological or medical response of a tissue system, animal or human that is being sought by a researcher or clinician, resulting in a beneficial effect for at least a statistically significant fraction of patients, such as an improvement of symptoms, a cure, a reduction in disease load, reduction in tumor mass or leukaemia cell numbers, improvement of peripheral blood cell counts, extension of life, or improvement in quality of life.

Day 1 of a 4 week treatment cycle is defined as that day on which the first dose of Volasertib administered.

Older patients with AML are biologically and clinically distinct from those who are younger. Although the prognosis of AML worsens with increasing age, older patients are generally considered as age 60 years or older. They are more likely to suffer early death and to exhibit therapeutic resistance. Increasing age is associated with factors predictive of early death, such as poor performance status or various comorbidities, and of treatment resistance (e.g. adverse cytogenetics, secondary AML or the MDR phenotype). Therefore, a substantial number of older AML patients are not considered for intensive treatment.

Although patients that are considered as ineligible for intensive treatment constitute a generally accepted subgroup of AML patients, no validated criteria are defined to judge a patient's eligibility for intensive treatment. The assessment of eligibility for intensive treatment is regularly done for every single patient based on the specialized physician's clinical experience and the comprehensive review of factors like patient age, AML cytogentics/molecular genetics, performance score, organ dysfunctions and comorbidities, as well as the patient's informed decision. In current practice, the final decision to treat intensively or not is made by the treating haematologist on a case by case basis. This approach is reflected in current practice guidelines (e.g. European LeukemiaNet (ELN) recommendations, NCCN guideline (National Comprehensive Cancer Network)).

Within the present invention the term “AML” is to be understood to encompass all forms of acute myeloid leukemia and related neoplasms according to the 2008 revision of theWorld Health Organization (WHO) classification of myeloid neoplasms and acute leukemia. These are:
Acute myeloid leukemia with recurrent genetic abnormalities
   AML with t(8;21)(q22;q22); RUNX1-RUNX1 T1
   AML with inv(16)(p13.1q22) or t(16;16)(p13.1;q22); CBFB-MYH11
   AML with t(9;11)(p22;q23); MLLT3-MLL
   AML with t(6;9)(p23;q34); DEK-NUP214
   AML with inv(3)(q21 q26.2) or t(3;3)(q21;q26.2); RPN1-EVI1
   AML (megakaryoblastic) with t(1;22)(p13;q13); RBM15-MKL1
   Provisional entity: AML with mutated NPM1
   Provisional entity: AML with mutated CEBPA
Acute myeloid leukemia with myelodysplasia-related changes
Therapy-related myeloid neoplasms
Acute myeloid leukemia, not otherwise specified
   AML with minimal differentiation
   AML without maturation
   AML with maturation
   Acute myelomonocytic leukemia
   Acute monoblastic/monocytic leukemia
   Acute erythroid leukemia
      Pure erythroid leukemia
      Erythroleukemia, erythroid/myeloid
   Acute megakaryoblastic leukemia
   Acute basophilic leukemia
   Acute panmyelosis with myelofibrosis
Myeloid sarcoma
Myeloid proliferations related to Down syndrome
   Transient abnormal myelopoiesis
   Myeloid leukemia associated with Down syndrome
Blastic plasmacytoid dendritic cell neoplasm

Within the present invention the term “MDS” is to be understood to encompass all forms of myelodysplastic/myeloproliferative neoplasms (MDS/MPN) and myelodysplastic syndromes according to the 2008 revision of theWorld Health Organization (WHO) classification of myeloid neoplasms and acute leukemia. These are:
Myelodysplastic/myeloproliferative neoplasms (MDS/MPN)
   Chronic myelomonocytic leukemia
   Atypical chronic myeloid leukemia, BCR-ABL1-negative
   Juvenile myelomonocytic leukemia
   Myelodysplastic/myeloproliferative neoplasm, unclassifiable
   Provisional entity: refractory anemia with ring sideroblasts and thrombocytosis
Myelodysplastic syndrome (MDS)
   Refractory cytopenia with unilineage dysplasia
   Refractory anemia
   Refractory neutropenia
   Refractory thrombocytopenia
   Refractory anemia with ring sideroblasts
   Refractory cytopenia with multilineage dysplasia
   Refractory anemia with excess blasts
   Myelodysplastic syndrome with isolated del(5q)
   Myelodysplastic syndrome, unclassifiable
   Childhood myelodysplastic syndrome
   Provisional entity: refractory cytopenia of childhood

In accordance with the present invention Volasertib may be administered by parenteral (e.g. intramuscular, intraperitoneal, intravenous, transdermal or subcutaneous injection), and may be formulated, alone or together, in suitable dosage unit formulations containing conventional non-toxic pharmaceutically acceptable carriers, adjuvants and vehicles appropriate for each route of administration. Dosage forms and formulations of both active ingredients suitable within the present invention are known in the art. For instance, such dosage forms and formulations include those disclosed for Volasertib in WO 2006/018221.

In accordance with the present invention Decitabine may be administered by parenteral (e.g. intramuscular, intraperitoneal, intravenous, transdermal or subcutaneous injection, or implant), routes of administration and may be formulated, alone or together, in suitable dosage unit formulations containing conventional non-toxic pharmaceutically acceptable carriers, adjuvants and vehicles appropriate for each route of administration.

The following Examples serve to illustrate the invention without restricting it:

Cells

MV4;11 (CRL-9591) cells were obtained from ATCC. According to the Catalogue of Somatic Mutations in Cancer of the Wellcome Trust Sanger Institute, UK, this cell line carries a mutation in the FLT3 gene. Cells were cultured in T175 tissue culture flasks at 37° C. and 5% CO₂. The medium used was IMDM supplemented with 10% fetal calf serum, 1% NEAA, 1% sodium pyruvate and 1% glutamine.

Mice

Mice were 8-9 week-old athymic female BomTac: NMRI-Foxn1^{nu} purchased from Taconic, Denmark. After arrival in the animal facility, mice were allowed to adjust to ambient conditions for at least 3 days before they were used for experiments. They were housed in Macrolon® type II cages in groups of 5 under standardized conditions at 21.5±1.5° C. temperature and 55±10% humidity. Standardized diet (PROVIMI KLIBA) and autoclaved tap water were provided ad libitum.

Establishment of Tumors, Randomization

To establish subcutaneous tumors, MV4;11 cells were harvested and resuspended in PBS+5% FCS at 5×10⁷ cells/ml. 50 μl of the cell suspension containing 2.5×10⁶ cells was then injected subcutaneously into the right flank of the mice (1 site per mouse). Growth factor reduced BD Matrigel™ Matrix (BD Biosciences) was added to the cell suspension at a ratio of 1:1 before the injection. When tumors were well established and had reached a tumor volume of ˜90 mm³, mice were randomly distributed between the treatment and the vehicle control groups 12 days after injecting the cells.

Administration of Test Compounds

Volasertib (BI 6727) was dissolved in hydrochloric acid (0.1 N), diluted with 0.9% NaCl and injected intravenously into the tail vein. An administration volume of 10 ml per kg body weight was used. The solution was freshly made up each injection day.

Decitabine was dissolved in 0.9% NaCl and administered i.p. An administration volume of 10 ml per kg body weight was used.

The application solution was stored for several days at 4° C.

Monitoring Tumor Growth and Side Effects

The tumor diameter was measured three times a week with a caliper. The volume of each tumor [in mm³] was calculated according to the formula “tumor volume=length*diameter²*π/6”. To monitor side effects of treatment, mice were inspected daily for abnormalities and body weight was determined three times a week. Animals were sacrificed at the end of the study when the control tumors reached a size of approximately 1100 mm³ on average. In addition animals with tumor sizes exceeding 2000 mm³ were sacrificed early during the studies for ethical reasons.

### EXAMPLE 1

### Nude Mouse Xenograft Model Derived from Human AML Cell Line MV4;11

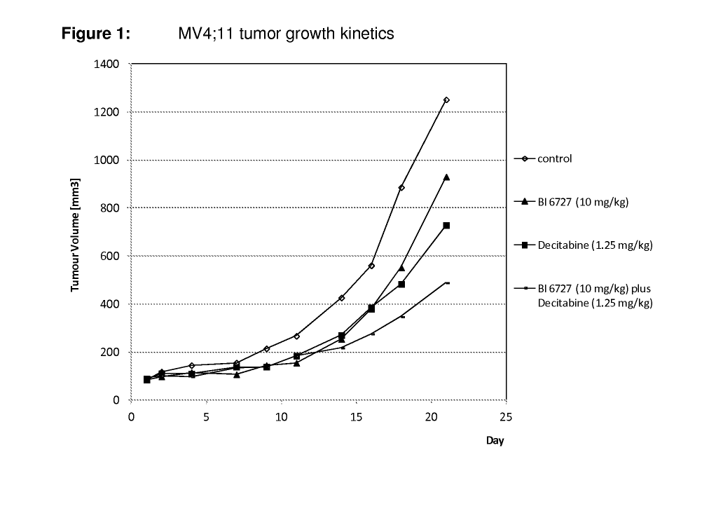

Results of an experiment comparing treatment of xenografts in mice with Volasertib alone (10 mg/kg, i.v.), administered once weekly, decitabine alone (1.25 mg/kg, i.p.), administered twice weekly on two consecutive days, and the combination of Volasertib/decitabine (10 mg/kg, i.v./1.25 mg/kg, i.p.) are shown in FIG. 1.

Animals were treated for 21 days.

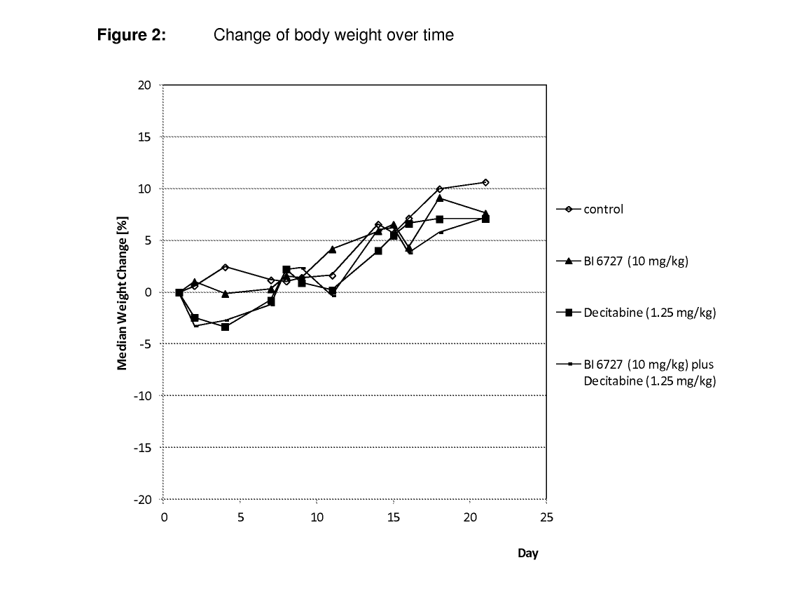

A combination of 10 mg/kg Volasertib i.v. once a week plus 1.25 mg/kg decitabine i.p. twice a week on consecutive days (T/C=40%; T/C: ratio of median tumor volume of treated vs. control tumors) showed reduced tumor growth compared to either single agent treatment (Volasertib: T/C=76%; decitabine: T/C=59%). Beneficial side effect profile was demonstrated as body weight gain in the combination group was comparable to single-agent Volasertib or single-agent decitabine as shown in FIG. 2.

### EXAMPLE 2

### Nude Mouse Xenograft Model Derived from Human AML Cell Line MV4;11

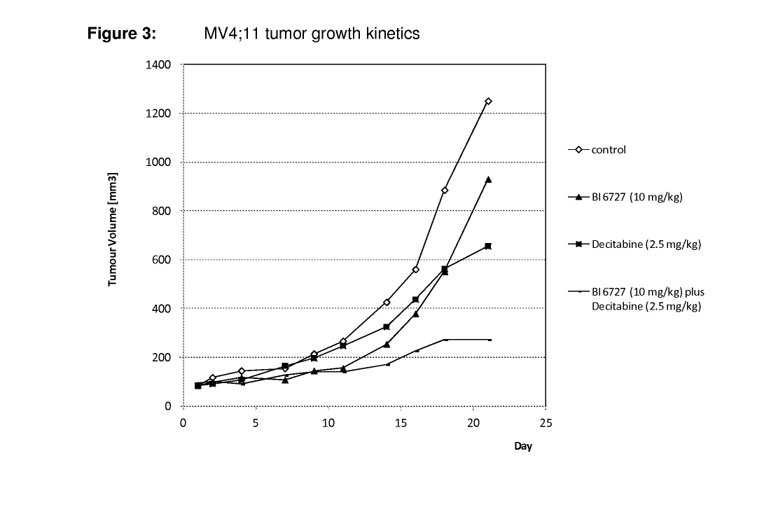

Results of an experiment comparing treatment of xenografts in mice with Volasertib alone (10 mg/kg, i.v.), administered once weekly, decitabine alone (2.5 mg/kg, i.p.), administered twice weekly on two consecutive days, and the combination of Volasertib/decitabine (10 mg/kg, i.v./2.5 mg/kg, i.p.) are shown in FIG. 3.

Animals were treated for 21 days.

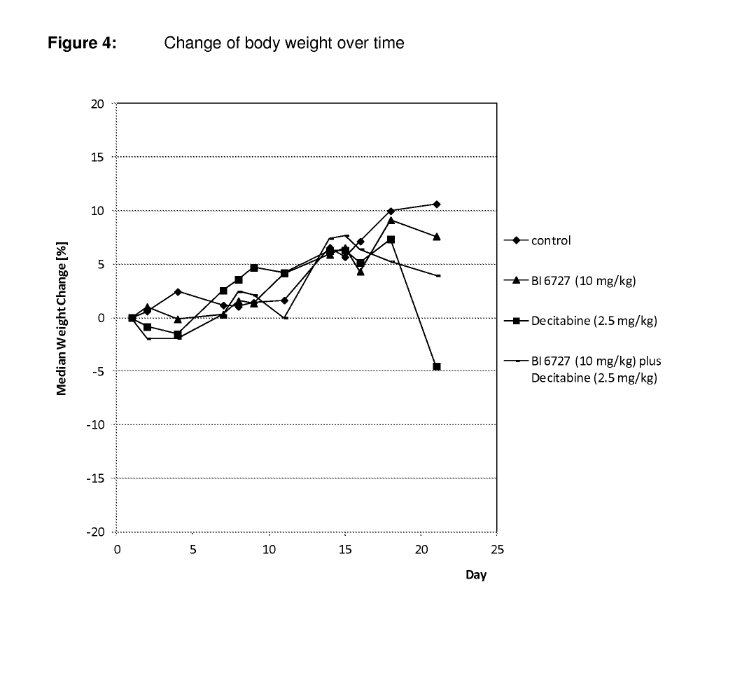

A combination of 10 mg/kg Volasertib i.v. once a week plus 2.5 mg/kg decitabine i.p. twice a week on consecutive days (T/C=22%) showed showed reduced tumor growth compared to either single agent treatment (Volasertib: T/C=76%; decitabine: T/C=53%). Beneficial side effect profile was demonstrated as body weight gain in the combination group was comparable to single-agent decitabine or single agent Volasertib as shown in FIG. 4.

### EXAMPLE 3

### Nude Mouse Xenograft Model Derived from Human AML Cell Line MV4;11

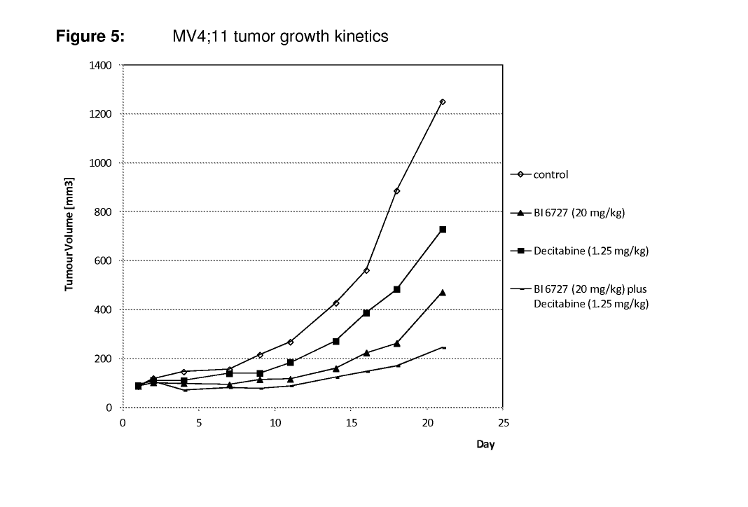

Results of an experiment comparing treatment of xenografts in mice with Volasertib alone (20 mg/kg, i.v.), administered once weekly, decitabine alone (1.25 mg/kg, i.p.), administered twice weekly on two consecutive days, and the combination of Volasertib/decitabine (20 mg/kg, i.v./1.25 mg/kg, i.p.) are shown in FIG. 5.

Animals were treated for 21 days.

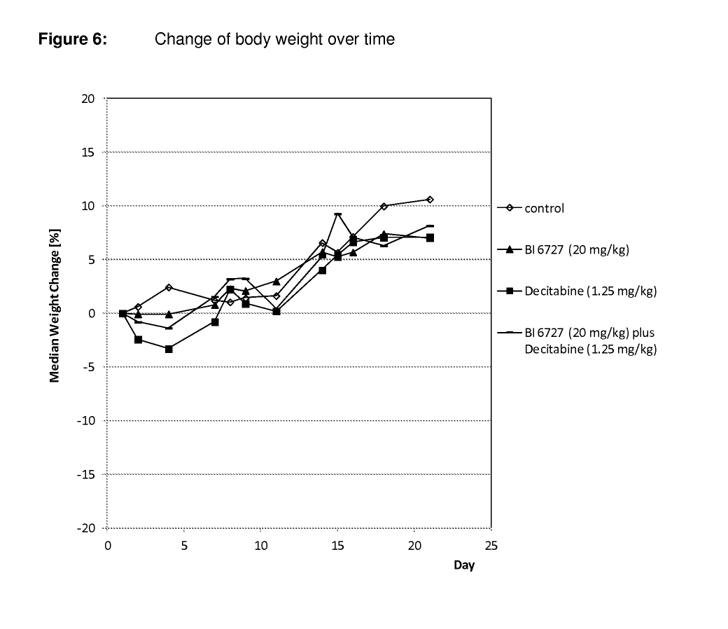

A combination of 20 mg/kg Volasertib i.v. once a week plus 1.25 mg/kg decitabine i.p. twice a week on consecutive days (T/C=20%) showed showed reduced tumor growth compared to either single agent treatment (Volasertib: T/C=38%; decitabine: T/C=59%,). Beneficial side effect profile was demonstrated as body weight gain in the combination group was comparable to single-agent Volasertib or single-agent decitabine as shown in FIG. 6.

1. A method of treating AML and/or MDS comprising administering to a patient a therapeutically effective amount of Volasertib optionally in the form of a pharmaceutically acceptable salt thereof or a hydrate thereof in combination with Decitabine, optionally in the form of a pharmaceutically acceptable salt thereof or a hydrate thereof, wherein both active ingredients are administered simultaneously, separately or sequentially.

2. The method according to claim 1, wherein Volasertib is administered in combination with Decitabine according to a dosage schedule (I) comprising or consisting of
a) administration of an effective amount of Volasertib on minimally one day during a 4 week treatment cycle and
b) administration of an effective amount of Decitabine on at least one day of the said 4 week treatment cycle.

3. The method according to claim 1, wherein Volasertib is administered in combination with Decitabine according to a dosage schedule (I) comprising or consisting of
a) administration of an effective amount of Volasertib on minimally two days during a 4 week treatment cycle and
b) administration of an effective amount of Decitabine on at least one day of the said 4 week treatment cycle.

4. The method according to claim 2 or 3, wherein 250 to 500 mg of Volasertib is administered per day of administration.

5. The method according to claim 2 or 3, wherein 5 to 50 mg/m² BSA of Decitabine are administered per day of administration.

6. The method according to claim 2 or 3, wherein Decitabine is administered on 5 days of the said 4 week treatment cycle.

7. The method according to claim 2 or 3, wherein Decitabine is administered on 10 days of the said 4 week treatment cycle.

8. The method according to claim 2 or 3, wherein the patient is older than 60 years.

9. A pharmaceutical composition comprising a therapeutically effective amount of Volasertib or a pharmaceutically acceptable salt thereof or a hydrate thereof, and a therapeutically effective amount of Decitabine or a pharmaceutically acceptable salt thereof or a hydrate thereof.

10. A pharmaceutical kit, comprising a first compartment which comprises a therapeutically effective amount of Volasertib or a pharmaceutically acceptable salt thereof or a hydrate thereof and a second compartment which comprises a therapeutically effective amount of Decitabine or a pharmaceutically acceptable salt thereof or a hydrate thereof.

11. A pharmaceutical composition or pharmaceutical kit according to claim 9 or 10 for simultaneous, separate or sequential use as medicament for treating AML and/or MDS.

12. A pharmaceutical combination, characterized in that it comprises Volasertib, optionally in the form of a pharmaceutically acceptable salt thereof or a hydrate thereof, and Decitabine, optionally in the form of a pharmaceutically acceptable salt thereof or a hydrate thereof, for simultaneous, separate or sequential use of the active ingredients as medicament for treating AML and/or MDS.
